(19) United States
(12) Patent Application Publication
(10) Pub. No.: US 2021/0282718 A1
(43) Pub. Date: Sep. 16, 2021

(54) IMPROVED SIGNAL QUALITY INDEX OF MULTICHANNEL BIO-SIGNAL USING RIEMANNIAN GEOMETRY

(71) Applicant: MENSIA TECHNOLOGIES, Chantepie Cedex (FR)

(72) Inventors: Quentin BARTHELEMY, Lyon (FR); Louis MAYAUD, Angoulême (FR)

(73) Assignee: MENSIA TECHNOLOGIES, Chantepie Cedex (FR)

(21) Appl. No.: 16/484,573

(22) PCT Filed: Dec. 15, 2017
(86) PCT No.: PCT/EP2017/083027

§ 371 Date: Aug. 8, 2019

(30) Foreign Application Priority Data
Dec. 15, 2016 (EP) 16204486.1

(51) Int. Cl. A61B 5/00; A61B 5/369

(52) U.S. Cl. CPC A61B 5/7221; A61B 5/369; A61B 5/725

(57) ABSTRACT

A system for computing a signal quality index from a multichannel bio-signal of a subject, the system including at least two sensors for acquiring one multichannel bio-signal of the subject; a memory including a reference covariance matrix and; a computing unit for computing a signal quality index, wherein the computing unit computes Riemannian distances between the reference covariance matrix and covariance matrices obtained from the multichannel bio-signal.

### FIELD OF INVENTION

The present invention pertains to the field of signal processing. In particular, the invention relates to a system for computing a signal quality index from a multichannel bio-signal of a subject in order to detect artifacts.

### BACKGROUND OF INVENTION

Bio-signals are a class of signals that are representations of underlying physiological processes. Bio-signals are particularly useful to monitor a patient's or subject's physiological state, diagnose illness or propose therapeutic or non-therapeutic methods.

A particularity of this class of signals is its high sensibility to environmental noise and other artifacts. Common artifacts are often of large amplitude compared to the bio-signal itself, which is typically a signal of rather small amplitude. Contamination by artifacts significantly reduces the utility of a bio-signal by giving a very low signal-to-noise ratio. During these artifacts, it is difficult to extract the information from noise. There is therefore a strong need in the medical field for signal processing techniques that remove artifacts from bio-signal prior to its interpretation.

One example of bio-signal is electroencephalogram, a measure of electrical brain activity. Electroencephalogram is very susceptible to various artifacts such as eye movement, eye blink, heart and muscle activities, electrode pop, swallowing, head and body movements.

A solution consists in detecting artifacts to suspend the processing: this approach avoids making bad signal processing. Said solution requires designing a quality index and a threshold for the detection of artifacts.

It is known to use covariance matrices which are processed with Riemannian geometry to detect artifacts from multichannel electroencephalogram signals (A. Barachant, A. Andreev, and M. Congedo, “The Riemannian potato: an automatic and adaptive artifact detection method for online experiments using Riemannian geometry”, in Proceedings of TOBI Workshop IV, 2013, pp.19-20).

The method introduced by Barachant et al. computes the Riemannian distance between a current covariance matrix and an online covariance matrix mean. An arithmetic z-score of the distance is then computed. If the z-score is lower than a given threshold, the signal is considered as normal and covariance matrix mean is updated; if the z-score is higher than the threshold, the signal is considered as artifacted. The z-score may thus be considered as a signal quality index.

The method disclosed by Barachant et al. is applied on all the channels C of the EEG signal, which give a feature space with

$$C \times ( \frac{C + 1}{2} ) .$$

However, if the dimensions of the covariance matrix become too large, even a huge variation in one dimension (representative of an artifact) disappears in the noise of the other dimensions: the distance between the current covariance matrix and the reference covariance matrix does not increase significantly and does not exceed the threshold. Consequently, when the number of channels C of the bio-signal is important (just as the number of dimensions of the covariance matrix), the detection of artifacts lacks efficiency. This phenomenon is accentuated with spatiofrequential covariance matrix: for F frequency bands, it gives a

$$F \times C \times ( \frac{C + 1}{2} ) .$$

feature space with

It is therefore an object of the present invention to provide an improved method for detecting all types of artifacts, even those occurring on a limited (e.g. one) number of channels among multichannel bio-signal.

### SUMMARY

To that end, the present invention provides a system for computing a signal quality index from multichannel bio-signal of a subject wherein said multichannel bio-signal is divided into multiple selected bio-signals based on subsets of channels and/or on frequency bands dedicated to the detection of specific artifacts. Said method is more sensitive to the variation of each channel.

The present invention thus relates to a system for computing a signal quality index from a multichannel bio-signal of a subject, said system comprising:
at least two sensors for acquiring one multichannel bio-signal of said subject on a current epoch t;
a memory comprising at least one reference covariance matrix Eₜ, at least one first threshold and the mean and standard deviation of Riemannian distances from the previous epochs; and
a computing unit for computing a signal quality index;

wherein the memory is connected to the computing unit and the at least two sensors are connected to the computing unit; and wherein the computing unit:
selects at least one subset of channels from said multichannel bio-signal or applies at least one frequency filtering to said multichannel bio-signal in order to get at least two selected bio-signals on the current epoch t;
for each of said at least two selected bio-signals on the current epoch t:
   computes a current covariance matrix Σ;
   computes the current Riemannian distance d between said current covariance matrix Σ and the at least one reference covariance matrix 9̅3̅ ₜ;
   computes an intermediary index based on a normalization of the current Riemannian distance d by the mean and the standard deviation of the previous Riemannian distances obtained on the previous epochs for which the intermediary index was inferior to the first threshold; and
computes a signal quality index based at least on the intermediary index for each of said at least two selected bio-signals.

According to one embodiment, the multichannel bio-signal is filtered in multiple frequency bands and concatenated by the computed unit and the computing unit computes a current spatiofrequential covariance matrix for each of said at least two selected bio-signals. According to one embodiment, the normalization for computing the intermediary index uses the geometric mean and the geometric standard deviation, more preferably the online geometric mean and the online geometric standard deviation. According to one embodiment, the normalization for computing the intermediary index is a z-score, preferably a geometric z-score.

According to one embodiment, for each of said at least two selected bio-signals on the current epoch t the computing unit updates the reference covariance matrix with the current covariance matrix if the intermediary index is inferior to the first threshold. According to one embodiment, the reference covariance matrix is updated as follows:

$${\sum }_{t + 1}^{\_} = {\overline{\sum }}_{t}^{\frac{1}{2}} {( {\overline{\sum }}_{t}^{\frac{1}{2}} \sum {\overline{\sum }}_{t}^{\frac{1}{2}} )}^{α} {\overline{\sum }}_{t}^{\frac{1}{2}}$$

where α ∈ [0,1] is a coefficient defining the speed of adaptation; it can be a constant or chosen as 1/(t+1). According to one embodiment, the multichannel bio-signal is an EEG signal. According to one embodiment, the multichannel bio-signal is an EEG signal and the computing unit selects a subset of channels representative of an EEG signal in a frontal area in order to get at least one selected bio-signal. According to one embodiment, the multichannel bio-signal is an EEG signal and the computing unit selects a subset of channels representative of two sensors in order to get at least one selected bio-signal.

According to one embodiment, wherein the multichannel bio-signal is an EEG signal and the computing unit selects a subset of channels representative of an EEG signal in an occipital area in order to get at least one selected bio-signal. According to one embodiment, the multichannel bio-signal is an EEG signal and the computing unit selects a subset of channels representative of an EEG signal in a temporal area in order to get at least one selected bio-signal.

According to one embodiment, the at least one selected bio-signal is filtered by the computing unit in low frequencies, preferably in the frequency band ranging from 1 Hz to 20 Hz or in high frequencies, preferably in the frequency band ranging from 55 Hz to 95 Hz. According to one embodiment, at least one selected bio-signal is obtained by frequency filtering the multichannel bio-signal in at least one frequency band which is not of interest for the underlying physiological process, such as a frequency band ranging from 1 Hz to 3 Hz or from 32 Hz to 45 Hz.

According to one embodiment, the memory further comprises a second threshold and the computing unit identifies artifacts if the signal quality index is lower than the second threshold. According to one embodiment, the signal quality index is computed using Fisher's combined probability test, preferably a right-tailed Fisher's combined probability test.

The present invention also relates to a method for computing a signal quality index from a multichannel bio-signal of a subject comprising iteratively the following steps:
i. obtaining one multichannel bio-signal from the subject on a current epoch t;
ii. selecting a subset of channels from said multichannel bio-signal or applying a frequency filtering to said multichannel bio-signal in order to get at least two selected bio-signals on the current epoch t;
iii. for each of said at least two selected bio-signals on the current epoch t:
   computing a current covariance matrix Σ;
   computing the current Riemannian distance d between said current covariance matrix Σ and a reference covariance matrix Σ̅ₜ;
   computing an intermediary index based on a normalization of the current Riemannian distance d by the mean and standard deviation of the previous Riemannian distances obtained on the previous epoch for which the intermediary index was inferior to a first threshold;
   preferably updating the reference covariance matrix with the current covariance matrix if the intermediary index is inferior to a first threshold;
   preferably updating the mean and standard deviation of the previous Riemannian distances if the intermediary index is inferior to a first threshold;
iv. computing a signal quality index based at least on the intermediary index for each of said at least two selected bio-signals; and
v. repeating steps i) to iv).

### DEFINITIONS

In the present invention, the following terms have the following meanings:
The term “about” is used herein to mean approximately, roughly, around, or in the region of. When the term “about” is used in conjunction with a numerical range, it modifies that range by extending the boundaries above and below the numerical values set forth. The term “about” is used herein to modify a numerical value above and below the stated value by a variance of 20 percent, preferably of 5 percent.
“Bio-signal” refers herein to any signal in subjects that can be continually measured and monitored. Bio-signal refers especially to any biological parameter which can be measured by an instrument which converts a physical measure (light, pressure, electricity, radio-signal . . . ) into an analogous signal (in volts) and which is then digitalized.
“Computing unit” refers to a computer-based system or a processor-containing system or other system which can fetch and executes the instructions of a computer program.
“Electrode” or “sensor” refers to a conductor used to establish electrical contact with a nonmetallic part of a circuit. For instance, EEG electrodes are small metal discs usually made of stainless steel, tin, gold, silver covered with a silver chloride coating; they are placed on the scalp in specific positions.
“Electroencephalogram” or “EEG” refers to the record of the electrical activity of the brain of a subject, made by electrodes applied on the scalp.
“Epoch” or “time-window” refers to a determined period during which bio-signal is recorded. Epochs can be overlapped.
“Fisher's combined probability test” refers to a method used to combine p-value from several tests. See R. Fisher, Statistical methods for research workers, Genesis Publishing Pvt Ltd, 1925.
“Index” refers to any value obtained or computed according to the present invention. In the sense of the present invention, the index is a value understandable by a subject.
“Neural signal” refers herein to the bio-signal obtained by measuring neural activity. Said neural activity may be measured by: deep brain electrodes; electrocorticography (ECoG); electroencephalography (EEG); magnetoencephalography (MEG); magnetic resonance imaging (MRI): diffusion MRI, perfusion MRI, functional MRI (fMRI); near-infrared spectroscopy (NIRS); positron emission tomography (PET); or stereoelectroencephalography (SEEG).
“Normalization” refers to a mathematical process to define values in an expected range: normalized values can be bounded, or normalized values can follow a normal/Gaussian distribution.
“Online” refers to methods that work on a sample-by-sample basis. However, the EEG acquisition is generally carried out by buffered blocks. These methods are different from “block-online” in that the block size may be very small (typically, 10 to 50ms for online, instead of 1 to 4s for block-online).
“Riemannian manifold” refers to a differentiable topological space that is locally homeomorphic to a Euclidean space, and over which a scalar product is defined that is sufficiently regular. The scalar product makes it possible to define a Riemannian geometry on the Riemannian manifold.
“Subject” refers to a mammal, preferably a human. In the sense of the present invention, a subject may be a patient, i.e. a person receiving medical attention, undergoing or having underwent a medical treatment, or monitored for the development of a disease.
“Symmetric positive definite (SPD) matrix” refers to a square matrix that is symmetrical about its diagonal, i.e. Σ(i, j)=Σ(j, i), and that has eigenvalues that are strictly positive. An SPD matrix of dimensions C×C has

$$C \times ( \frac{C + 1}{2} )$$

independent elements; it may therefore be locally approximated by a Euclidean space of

$$C \times ( \frac{C + 1}{2} )$$

dimensions. It is possible to show that the SPD space has the structure of a Riemannian manifold. It is known that covariance matrices are symmetric positive definite matrices.
“Threshold” refers to a reference value to which a current value is compared.

### DETAILED DESCRIPTION

This invention relates to a system for computing a signal quality index from a multichannel bio-signal of a subject.

The system comprises at least two sensors for acquiring one multichannel bio-signal of said subject.

According to one embodiment, the multichannel bio-signal is a multichannel neural signal.

According to one embodiment, the multichannel neural signal is a multichannel EEG signal. In said embodiment, various types of suitable headsets or electrodes systems or sensors systems are available for acquiring such a EEG signal. Examples includes, but are not limited to: Epoc headset commercially available from Emotiv, Mindset headset commercially available from Neurosky, Versus headset commercially available from SenseLabs, DSI 6 headset commercially available from Wearable sensing, Xpress system commercially available from BrainProducts, Mobita system commercially available from TMSi, Porti32 system commercially available from TMSi, ActiChamp system commercially available from BrainProducts and Geodesic system commercially available from EGI.

According to one embodiment, the multichannel EEG signal is acquired using a set of sensors (also referred to as electrodes). According to one embodiment, the multichannel EEG signal is acquired using 8 electrodes, 16 electrodes, 24 electrodes, 32 electrodes, 64 electrodes, 128 electrodes or 256 electrodes. Said electrodes are preferably positioned according to the international 10-20 system.

The overall acquisition time of the multichannel bio-signal is subdivided into periods, known in the art as epochs. Each epoch is associated with a matrix X representative of the bio-signal acquired during said epoch.

Therefore, the system comprises at least two sensors for acquiring one multichannel bio-signal of said subject on successive epochs.

According to one embodiment, in order to ensure online (or real-time) processing, successive epochs are overlapped.

A multichannel bio-signal X ∈ [symbol] ^{C×N} is composed of C channels, electrodes or sensors and N time samples. For example, a subject is fitted with C electrodes for neural signal acquisitions. Each electrode c=1 . . . C delivers a signal X_{c}(n) as a function of time. The signal is sampled so as to operate in discrete time: X(c, n)=X_{c}(n), and then digitized. This produced a matrix representation of the neural signal X ∈ [symbol] ^{C×N}.

According to one embodiment, the multichannel bio-signal is pre-processed, such as for instance centered, filtered, cleaned and/or denoised.

The system further comprises a memory and a computing unit. The memory is connected to the computing unit. The at least two sensors are connected to the computing unit for delivering the multichannel bio-signal.

The computing unit is configured to obtain at least two selected bio-signals from the original multichannel bio-signal either by selecting a subset of channels from the multichannel bio-signal or by frequency filtering the multichannel bio-signal.

Consequently, the computing unit selects at least one subset of channels from said multichannel bio-signal or applies at least one frequency filtering to said multichannel bio-signal in order to get at least two selected bio-signals on the successive epochs.

According to one embodiment wherein the computing unit selects at least one subset of channels from said multichannel bio-signal, the selected bio-signal comprises one channel According to one embodiment wherein the computing unit selects at least one subset of channels from said multichannel bio-signal, the selected bio-signal comprises a plurality of channels.

As explained hereabove, said selection and/or frequency filtering enables sensitive detection of specific artifacts.

According to an alternative embodiment, the computing unit is configured to obtain at least one selected bio-signal from the original multichannel bio-signal either by selecting a subset of channels from the multichannel bio-signal or by frequency filtering the multichannel bio-signal.

According to one embodiment wherein the multichannel bio-signal is an EEG signal, in order to detect electrode pops, the computing unit selects at least one subset of channel representative of two sensors. According to one embodiment wherein the multichannel bio-signal is an EEG signal, in order to detect electrode pop, the computing unit selects a plurality of selected bio-signals, each representative of two sensors. According to one embodiment wherein the multichannel bio-signal is an EEG signal, in order to detect electrode pop, the computing unit selects a selected bio-signal for each couple of two sensors.

According to one embodiment, said two sensors are:
two adjacent sensors;
two sensors symmetrical with respect to an axis, preferably with respect to an anteroposterior axis or a mediolateral axis, more preferably with respect to the anteroposterior axis or the mediolateral axis extending through the vertex; or
two sensors symmetrical with respect to a third sensor acting as a center of symmetry, preferably two sensors symmetrical with respect to the vertex.

According to one embodiment, said selected bio-signals are further band-pass filtered, preferably in low frequencies. According to one embodiment, selected bio-signals are band-pass filtered from about 1 to about 20 Hz.

According to one embodiment wherein the multichannel bio-signal is an EEG signal, in order to detect electrooculogram activities such as for instance eye blinks, the computing unit selects a subset of channels representative of an EEG signal in a frontal area (i.e. at least one of the selected bio-signal comprises a subset of channels representative of frontal sensors). According to one embodiment, said selected bio-signal is further band-pass filtered, preferably in low frequencies. According to one embodiment, selected bio-signal is band-pass filtered from about 1 to about 20 Hz.

According to one embodiment wherein the multichannel bio-signal is an EEG signal, in order to detect muscular artifacts such as for instance neck tension, the computing unit selects a subset of channels representative of an EEG signal in occipital area (i.e. at least one of the selected bio-signal comprises a subset of channel representative of occipital sensors). According to one embodiment, said selected bio-signal is further band-pass filtered, preferably in high frequencies. According to one embodiment, selected bio-signal is band-pass filtered from about 55 to about 95 Hz or from about 65 to about 95 Hz or from about 65 to about 115 Hz.

According to one embodiment wherein the multichannel bio-signal is an EEG signal, in order to detect muscular artifacts such as for instance jaw clenching, the computing unit selects a subset of channels representative of a temporal EEG signal (i.e. at least one of the selected bio-signal comprises a subset of channels representative of temporal sensors). According to one embodiment, said selected bio-signal is further band-pass filtered, preferably in high frequencies. According to one embodiment, selected bio-signal is band-pass filtered from about 55 to about 95 Hz (if powerline is 50 Hz), or from about 65 to about 115 Hz (if powerline is 60 Hz).

According to one embodiment wherein the multichannel bio-signal is an EEG signal, in order to detect multiple artifacts such as for instance head motion, the computing unit frequency filters the multichannel bio-signal in at least one frequency band which is not of interest for the underlying process (for example, in at least one frequency band which is not the alpha band from 8 to 12 Hz or the beta band from 12.5 to 30 Hz). According to one embodiment, the computing unit frequency filters the multichannel bio-signal in a frequency band ranging from about 1 to about 3 Hz and/or in the frequency band ranging from about 32 to about 45 Hz.

According to one embodiment, the selected bio-signals comprise any combination of the above-described selected bio-signals.

According to one embodiment, the computing unit obtains 2, 3, 4, 5, 10, 15 or 20 selected bio-signals from any combination of the above-described embodiments.

According to one embodiment, for each of said at least two selected bio-signals on the current epoch t the computing unit:
computes a current covariance matrix Σₜ, denoted Σ in the following to lighten notations;
computes the current Riemannian distance d between said current covariance matrix Σ and a reference covariance matrix \hat{Σ}ₜ; and
computes an intermediary index based on a normalization of the current Riemannian distance d by the mean and standard deviation of the previous Riemannian distances obtained on the previous epochs for which the intermediary index was inferior to a first threshold.

Said reference covariance matrix Σ̅ₜ, said first threshold and said mean and standard deviation of the Riemannian distances computed from previous epochs are stored in the memory.

According to one embodiment, the first threshold is ranging from 1.0 to 3.5, preferably 2.0. According to the Applicant, a low value provides a strict behavior with updates only on “very clean” epochs, a high value provides a more dynamic behavior, with more updates.

According to one embodiment, a first threshold is stored in the memory for each selected bio-signal.

According to one embodiment, a covariance matrix E is computed from the bio-signal as follows:

$$\sum = \frac{1}{N - 1} {X}^{T} X ,$$

will N≥C, where N is the number of samples in the epoch for each electrode and C the number of electrodes.

In another embodiment, the covariance matrix is computed using any method known by the skilled artisan, such as those disclosed in Barachant A. Commande robuste d'un effecteur par une interface cerveau-machine EEG asynchrone, PhD. Thesis, Université de Grenoble: FR, 2012.

According to one embodiment, the covariance matrix is a spatial covariance matrix. According to an alternative embodiment, the covariance matrix is a spatiofrequential covariance matrix. Said embodiment enables to be sensitive to specific frequency bands. Such sensitivity is of particular importance for neural signal, especially EEG signal.

According to one embodiment, the cut-off frequencies of the frequential filtering are chosen arbitrarily. According to another embodiment, the cut-off frequencies of the frequential filtering are optimized thanks to a preliminary frequential study.

According to one embodiment, in order to obtain a spatiofrequential covariance matrix, the bio-signal X ∈ [symbol] ^{C×N} is filtered in F frequency bands; thereby obtaining f=1 . . . F filtered signals X_{f} ∈ [symbol] ^{C×N}. An extended signal \tilde{X} ∈ [symbol] ^{CF×N} defined as the vertical concatenation of the filtered signals is computed as:

$$\tilde{X} = [ \begin{matrix}{X}_{1} \\ ⋮ \\ {X}_{f} \\ ⋮ \\ {X}_{F}\end{matrix} ] .$$

The spatiofrequential covariance matrix \tilde{Σ} ∈ [symbol] ^{CF×CF} is then computed as follows:

$$\tilde{\sum } = \frac{1}{N - 1} {\tilde{X}}^{T} \tilde{X} ,$$

with N≥CF where N is the number of samples in the epoch for each electrode, and C the number of electrodes.

According to one embodiment, the covariance matrix (either spatial or spatiofrequential) is normalized. According to one embodiment, the covariance matrix is trace-normalized, which makes its trace equal to 1:

$$\sum = \frac{\sum }{trace ( \sum )} .$$

According to one embodiment, the covariance matrix is determinant-normalized, which makes its determinant equal to 1:

$$\sum = \frac{\sum }{{det ( \sum )}^{1 / C}} .$$

The normalization step, especially the determinant normalization allows a session-to-session and/or a subject-to-subject transfer learning: it removes a part of variabilities from one recording to another.

Each covariance matrix associated with a given epoch is considered to be point of a Riemannian manifold. In order to position the neural activity of a subject relative to a reference, distance and means calculation are required. The metric used for covariance matrices has been detailed in Förstner W, Moonen B. A metric for covariance matrices. Quo vadis geodesia, pp. 113-128, 1999.

According to one embodiment, the Riemannian distance between two covariance matrices Σ₁ and Σ₂ may be defined as the affine-invariant distance:

$$d ( {\sum }_{1} , {\sum }_{2} ) = {( {\sum }_{c = 1}^{C} {log}^{2} {λ}_{c} ( {\sum }_{1} , {\sum }_{2} ) )}^{1 / 2}$$

with λ_{c}(Σ₁, Σ₂) the eigenvalues from det(λΣ₁−Σ₂)=0.

According to another embodiment, the Riemannian distance is computed using any other distances known by one skilled in the art, such as those described in Li Y, Wong K M. Riemannian Distances for Signal Classification by Power Spectral Density. IEEE Journal of selected topics in signal processing, vol.7, No.4, August 2013.

According to one embodiment, the Riemannian distances are estimated on the Riemannian manifold of symmetric positive definite matrices of dimensions equal to the dimensions of the covariance matrices.

According to one embodiment, the normalization for computing the intermediary index uses the mean μₜ and the standard deviation σₜ of the previous Riemannian distances, preferably computed in their geometric form, for instance as follows:

$${μ}_{t} = exp ( \frac{1}{2} {\sum }_{τ = 1}^{t} log ( {d}_{τ} ) )$$

$${σ}_{t} = exp ( \sqrt{\frac{1}{2} {\sum }_{τ = 1}^{t} {( log ( \frac{{d}_{τ}}{{μ}_{t}} ) )}^{2}} )$$

According to one embodiment, mean μₜ and the standard deviation σₜ can be online updated, for instance as follows:

$${μ}_{t + 1} = exp ( \frac{t}{t + 1} log ( {μ}_{t} ) + \frac{1}{t + 1} log ( {d}_{t + 1} ) )$$

$${σ}_{t + 1} = exp ( \sqrt{\frac{t}{t + 1} {( log ( {σ}_{t} ) )}^{2} + \frac{1}{t + 1} {( log ( \frac{{d}_{t + 1}}{{μ}_{t + 1}} ) )}^{2}} )$$

According to one embodiment, the intermediary index is defined as the normalization of the Riemannian distance providing a z-score, also called standard score.

According to one embodiment, the z-score is preferably computed in its geometric form, for instance as follows:

$${z}_{t} = \frac{log ( \frac{{d}_{t}}{{μ}_{t}} )}{log ( {σ}_{t} )}$$

According to one embodiment, for each of said at least two selected bio-signals on the current epoch t the computing unit updates the reference covariance matrix with the current covariance matrix if the intermediary index is inferior to the first threshold.

According to one embodiment, the reference covariance matrix is updated as follows:

$${\overline{\sum }}_{t + 1} = {\overline{\sum }}_{t}^{\frac{1}{2}} {( {\overline{\sum }}_{t}^{- \frac{1}{2}} \sum {\overline{\sum }}_{t}^{- \frac{1}{2}} )}^{\frac{1}{t + 1}} {\overline{\sum }}_{t}^{\frac{1}{2}}$$

According to one embodiment, update coefficient

$$\frac{1}{t + 1}$$

can De repiaceu Dy any inner coefficient α ∈ [0,1] defining the speed of the adaptation.

As detailed hereabove, the reference covariance matrix is updated only if the intermediary index is lower than the first thresholds. Said embodiment prevents from updating the reference covariance matrix with a bio-signal comprising artifacts.

According to one embodiment, the reference covariance matrix is initialized online on the first epochs of the recording: Σ̅ₜ₌₁=Σ₁, μ₁=1, σ₁=e. This type of online initialization provides an online dynamic process.

According to one embodiment, the reference covariance matrix is initialized offline during a calibration session, avoiding an online initialization on the first epochs, potentially containing artifacts due to the device installation and thus disturbing the efficiency of the detection. In this case, the initial value t₀ of t defines the importance given to the calibration (high value for a confident initialization): Σ̅ₜ₌ₜ_{₀}=Σ̅_{calibration}, μₜ_{₀}=μ_{calibration}, σₜ_{₀}=σ_{calibration}. This type of offline initialization provides an online semi-static process, and is suitable for session-to-session or subject-to-subject transfer learning.

The computing unit further computes a signal quality index based at least on the intermediary index for each of said at least two selected bio-signals.

According to one embodiment, the signal quality index is based on the combination of intermediary indices computed for each of said at least two selected bio-signals.

According to one embodiment, the signal quality index is further based on a temporary index computed from the original multichannel bio-signal.

According to one embodiment, the signal quality index is computed using Fisher's combined probability test, preferably a right-tailed Fisher's combined probability test.

According to one embodiment, the intermediary indices are combined in a single p-value with a right-tailed Fisher's combined probability test.

According to one embodiment, considering i=1 . . . I intermediary indices, p-values pᵢ associated to each z-score zᵢ is defined as:

pᵢ=1− [symbol] (zᵢ)

wherein cdf_{N}_{_{0.1}} is the cumulative distribution function of the normal distribution N_{0.1} with mean equals to 0 and standard deviation equals to 1.

According to one embodiment, said p-values are then combined as follows:

$$s = - 2 {\sum }_{i = 1}^{I} log ( {p}_{i} ) ∼ {X}_{2 I}^{2}$$

According to one embodiment, the signal quality index is finally computed as follows:

p=1cdf_{X}_{_{2I}}_{²}(s)

wherein cdf_{X}_{_{2I}}_{²} is the cumulative distribution function of the chi-square distribution X_{2I}² with 2I degrees of freedom.

According to another embodiment, intermediary indices can be combined in a signal quality index using the Stouffer's method; the Edgington method; or the squared z-scores method.

According to one embodiment, the memory further comprises a second threshold.

According to one embodiment, the computing unit identifies artifacts if the signal quality index is lower than the second threshold. According to one embodiment, the computing unit considers the signal as “clean” if the signal quality index is higher than the second threshold.

According to one embodiment, the second threshold is ranging from 0.001 and 0.05, preferably 0.01.

The present invention further relates to a method for computing a signal quality index from a multichannel bio-signal of a subject comprising iteratively the following steps:
i. obtaining one multichannel bio-signal from the subject on a current epoch t;
ii. selecting a subset of channels from said multichannel bio-signal or applying a frequency filtering to said multichannel bio-signal in order to get at least two selected bio-signals on the current epoch t;
iii. for each of said at least two selected bio-signals on the current epoch t:
   computing a current covariance matrix Σₜ, denoted Σ to lighten notations;
   computing the current Riemannian distance d between said current covariance matrix Σ and a reference covariance matrix Σ̅ₜ;
   computing an intermediary index based on a normalization of the current Riemannian distance d by the mean and standard deviation of the previous Riemannian distances obtained on the previous epoch for which the intermediary index was inferior to a first threshold;
   preferably updating the reference covariance matrix Σ̅ₜ with the current covariance matrix if the intermediary index is inferior to a first threshold;
   preferably updating the mean μₜ and standard deviation σₜ of the previous Riemannian distances if intermediary index is inferior to a first channel; and
iv. computing a signal quality index combining at least the intermediary index for each of said at least two selected bio-signals;
v. repeating steps i) to iv).

According to one embodiment, the selection of a subset of channels from said multichannel bio-signal or the application of a frequency filtering to said multichannel bio-signal is implemented as detailed hereabove depending of the artifacts to be detected.

According to one embodiment, said method is a computer-implemented method.

According to one embodiment, the present invention also relates to computer program product comprising instructions which, when the program is executed by a computer, cause the computer to carry out the steps of the method according to the present invention.

According to one embodiment, the present invention also relates to a computer-readable storage medium comprising instructions which, when executed by a computer, cause the computer to carry out the steps of the method of the present invention.

While various embodiments have been described, the detailed description is not to be construed as being limited hereto. Various modifications can be made to the embodiments by those skilled in the art without departing from the true spirit and scope of the disclosure as defined by the claims.

### EXAMPLES

The present invention is further illustrated by the following examples.

### Example 1

In said example, multichannel EEG signal is recorded using 8 electrodes Fpz, F3, Fz, F4, C3, Cz, C4 and Pz positioned according to the 10-20 system.

In said example the following selected bio-signals are used:

In order to detect electrode pops, the computing unit obtains 4 selected bio-signals by selecting the following channels:
Fpz and Fz;
F3 and F4;
C3, C4; and
Cz and Pz.

Said selected bio-signals comprises EEG signal recorded from adjacent electrodes. Said selected bio-signals are further filtered using a band-pass filter from 1 to 20 Hz.

In order to detect multiple artifacts such as for instance head motion, the computing unit obtains a selected bio-signal by frequency filtering all the channels in the frequency band ranging from 1 to 3 Hz and from 32 to 45 Hz.

### Example 2

In said example, multichannel EEG signal is recorded using 8 electrodes Fpz, F3, Fz, F4, C3, Cz, C4 and Pz positioned according to the 10-20 system.

In said example the following selected bio-signals are used:

In order to detect eye blinks, and more generally all electro-oculogram activities the computing unit obtains a selected bio-signal by selecting the following channels: Fpz, F3, Fz and F4, representative of frontal electrodes. Said selected bio-signal is further filtered using a band-pass filter from 1 to 20 Hz.

In order to detect multiple artifacts such as for instance head motion, the computing unit obtains a selected bio-signal by frequency filtering all the channels in the frequency band ranging from 1 to 3 Hz and from 32 to 45 Hz.

### Example 3

In said example, multichannel EEG signal is recorded using 8 electrodes Fpz, F3, Fz, F4, C3, Cz, C4 and Pz positioned according to the 10-20 system.

In said example the following selected bio-signals are used:

In order to detect electrode pops, the computing unit obtains 4 selected bio-signals by selecting the following channels:
Fpz and Fz;
F3 and F4;
C3, C4; and
Cz and Pz.

Said selected bio-signals comprises EEG signal recorded from adjacent electrodes. Said selected bio-signals are further filtered using a band-pass filter from 1 to 20 Hz.

In order to detect eye blinks, and more generally all electro-oculogram activities the computing unit obtains a selected bio-signal by selecting the following channels: Fpz, F3, Fz and F4, representative of frontal electrodes. Said selected bio-signal is further filtered using a band-pass filter from 1 to 20 Hz.

In order to detect muscular artifact, such as for instance jaw clenching the computing unit obtains a selected bio-signal by selecting the following channel: Fpz, F3, F4, C3 and C4 representative of electrodes closest to muscles. Said selected bio-signal is further filtered using a band-pass filter from 65 to 95 Hz.

In order to detect multiple artifacts such as for instance head motion, the computing unit obtains a selected bio-signal by frequency filtering all the channels in the frequency band ranging from 1 to 3 Hz and from 20 to 48 Hz.

1-17. (canceled)

18. A system for computing a signal quality index from a multichannel bio-signal of a subject, said system comprising:
at least two sensors for acquiring one multichannel bio-signal of said subject on a current epoch t;
a memory comprising at least one reference covariance matrix Σ̅ₜ, at least one first threshold and the mean and standard deviation of Riemannian distances from the previous epochs; and
a computing unit for computing a signal quality index;
wherein the memory is connected to the computing unit and the at least two sensors are connected to the computing unit; and
wherein the computing unit:
selects at least a first subset of channels from said multichannel bio-signal and/or applies at least one frequency filtering to said multichannel bio-signal from the first subset in order to get at least a first selected bio-signal on the current epoch t;
selects at least a second subset of channels from said multichannel bio-signal and/or applies at least one frequency filtering to said multichannel bio-signal from the second subset in order to get at least a second selected bio-signal on the current epoch t;
for each of said at least the first and the second selected bio-signals on the current epoch t:
computes a current covariance matrix Σ;
computes the current Riemannian distance d between said current covariance matrix Σ and the at least one reference covariance matrix Σ̅ₜ;
computes an intermediary index based on a normalization of the current Riemannian distance d by the mean and the standard deviation of the previous Riemannian distances obtained on the previous epochs for which the intermediary index was inferior to the first threshold; and
computes a signal quality index based at least on the intermediary index for each of said at least two selected bio-signals.

19. The system according to claim 18, wherein the multichannel bio-signal is filtered in multiple frequency bands and concatenated by the computed unit and wherein the computing unit computes a current spatiofrequential covariance matrix for each of said at least two selected bio-signals.

20. The system according to claim 18, wherein the normalization for computing the intermediary index uses the geometric mean and the geometric standard deviation.

21. The system according to claim 20, wherein the normalization for computing the intermediary index is a z-score.

22. The system according to claim 18, wherein for each of said at least two selected bio-signals on the current epoch t the computing unit updates the reference covariance matrix with the current covariance matrix if the intermediary index is inferior to the first threshold.

23. The system according to claim 22, wherein the reference covariance matrix is updated as follows:
$${\sum }_{t + 1} = {\sum }_{t}^{\frac{1}{2}} {( {\sum }_{t}^{\frac{1}{2}} \sum {\sum }_{t}^{- \frac{1}{2}} )}^{\frac{1}{t + 1}} {\sum }_{t}^{\frac{1}{2}}$$

24. The system according to claim 18, wherein the multichannel bio-signal is an EEG signal and the computing unit selects a subset of channels representative of an EEG signal in a frontal area in order to get at least one selected bio-signal.

25. The system according to claim 18, wherein the multichannel bio-signal is an EEG signal and the computing unit selects a subset of channels representative of two sensors in order to get at least one selected bio-signal.

26. The system according to claim 18, wherein the multichannel bio-signal is an EEG signal and the computing unit selects a subset of channels representative of an EEG signal in an occipital area in order to get at least one selected bio-signal.

27. The system according to claim 18, wherein the multichannel bio-signal is an EEG signal and the computing unit selects a subset of channels representative of an EEG signal in a temporal area in order to get at least one selected bio-signal.

28. The system according to claim 18, wherein the at least one selected bio-signal is filtered by the computing unit in low frequencies or in high frequencies.

29. The system according to claim 18, wherein at least one selected bio-signal is obtained by frequency filtering the multichannel bio-signal in at least one frequency band which is not of interest for the underlying physiological process.

30. The system according to claim 18, wherein the memory further comprises a second threshold and the computing unit identifies artifacts if the signal quality index is lower than the second threshold.

31. The system according to claim 18, wherein the signal quality index is computed using Fisher's combined probability test.

32. A method for computing a signal quality index from a multichannel bio-signal of a subject comprising iteratively the following steps:
obtaining one multichannel bio-signal from the subject on a current epoch t;
selecting a subset of channels from said multichannel bio-signal or applying a frequency filtering to said multichannel bio-signal in order to get at least two selected bio-signals on the current epoch t;
for each of said at least two selected bio-signals on the current epoch t:
computing a current covariance matrix Σ;
computing the current Riemannian distance d between said current covariance matrix Σ and a reference covariance matrix Σ̅ₜ;
computing an intermediary index based on a normalization of the current Riemannian distance t by the mean and standard deviation of the previous Riemannian distances obtained on the previous epoch for which the intermediary index was inferior to a first threshold; and
computing a signal quality index based at least on the intermediary index for each of said at least two selected bio-signals; and repeating steps i) to iv).

33. A method according to claim 32, wherein the method further comprises updating the reference covariance matrix Σ̅ₜ, with the current covariance matrix if the intermediary index is inferior to a first threshold.

34. A method according to claim 32, wherein the method further comprises updating the mean μₜ and standard deviation σₜ of the previous Riemannian distances if intermediary index is inferior to the first threshold.
